(19) United States
(12) Patent Application Publication
(10) Pub. No.: US 2017/0296380 A1
(43) Pub. Date: Oct. 19, 2017

(54) Heated Mask Assembly

(71) Applicants: Randy G. Barger, Pittsburgh, PA (US); Vicki A. Barger, Pittsburgh, PA (US)

(72) Inventors: Randy G. Barger, Pittsburgh, PA (US); Vicki A. Barger, Pittsburgh, PA (US)

(21) Appl. No.: 15/131,620

(22) Filed: Apr. 18, 2016

(51) Int. Cl. A61F 7/02; A61F 7/00

(52) U.S. Cl. CPC A61F 7/02; A61F 2007/023; A61F 2007/0003; A61F 2007/0206

(57) ABSTRACT

A heated mask assembly includes a pouch that may be worn on a user's face. The pouch is comprised of a fluid absorbent material such as cotton or the like. Thus, the pouch may be soaked with a heated fluid. A heat pack is removably positioned in the pouch. The heat pack may be heated thereby facilitating the heat pack to provide heat therapy to the user's face when the pouch is worn. The heat pack provides heat therapy to the user's sinus cavities. A strap is coupled to the pouch. The strap may be wrapped around the user's head thereby facilitating the pouch to be retained on the user's face.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art including information disclosed under 37 CFR 1.97 and 1.98

The disclosure and prior art relates to mask devices and more particularly pertains to a new mask device for providing heat therapy to sinuses.

### BRIEF SUMMARY OF THE INVENTION

An embodiment of the disclosure meets the needs presented above by generally comprising a pouch that may be worn on a user's face. The pouch is comprised of a fluid absorbent material such as cotton or the like. Thus, the pouch may be soaked with a heated fluid. A heat pack is removably positioned in the pouch. The heat pack may be heated thereby facilitating the heat pack to provide heat therapy to the user's face when the pouch is worn. The heat pack provides heat therapy to the user's sinus cavities. A strap is coupled to the pouch. The strap may be wrapped around the user's head thereby facilitating the pouch to be retained on the user's face.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a heated mask assembly according to an embodiment of the disclosure.

FIG. 2 is an exploded perspective view of an embodiment of the disclosure.

FIG. 3 is a front view of an embodiment of the disclosure.

FIG. 4 is a cross sectional view taken along line 3 of an embodiment of the disclosure.

FIG. 5 is a perspective in-use view of an embodiment of the disclosure.

### DETAILED DESCRIPTION OF THE INVENTION

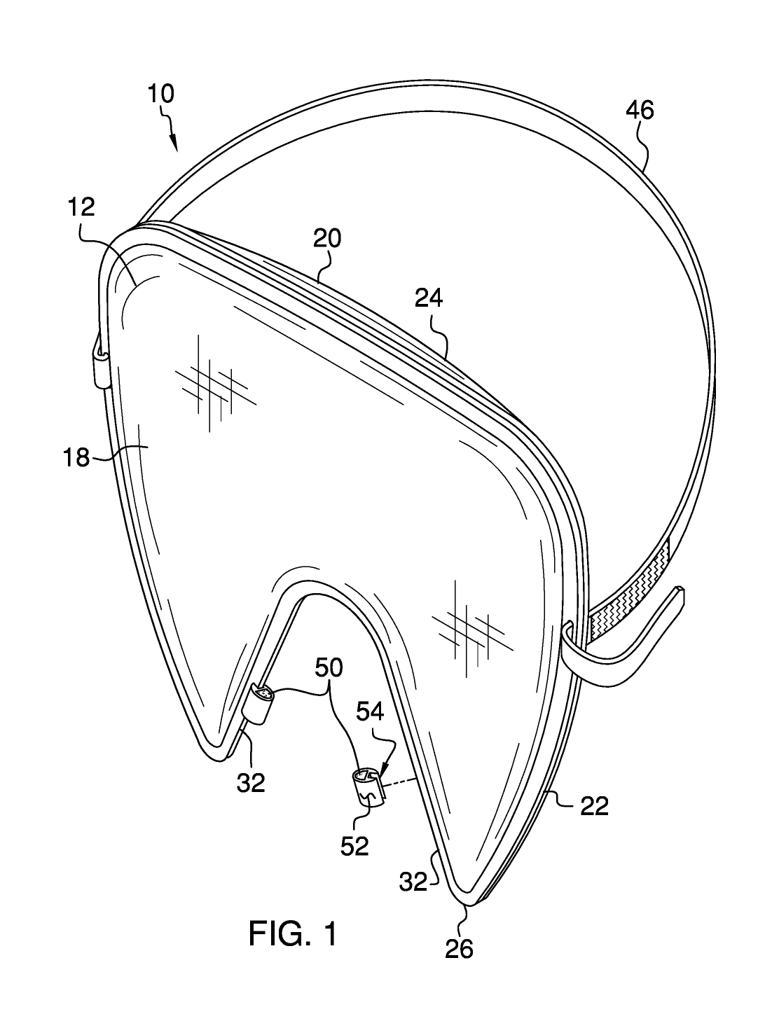

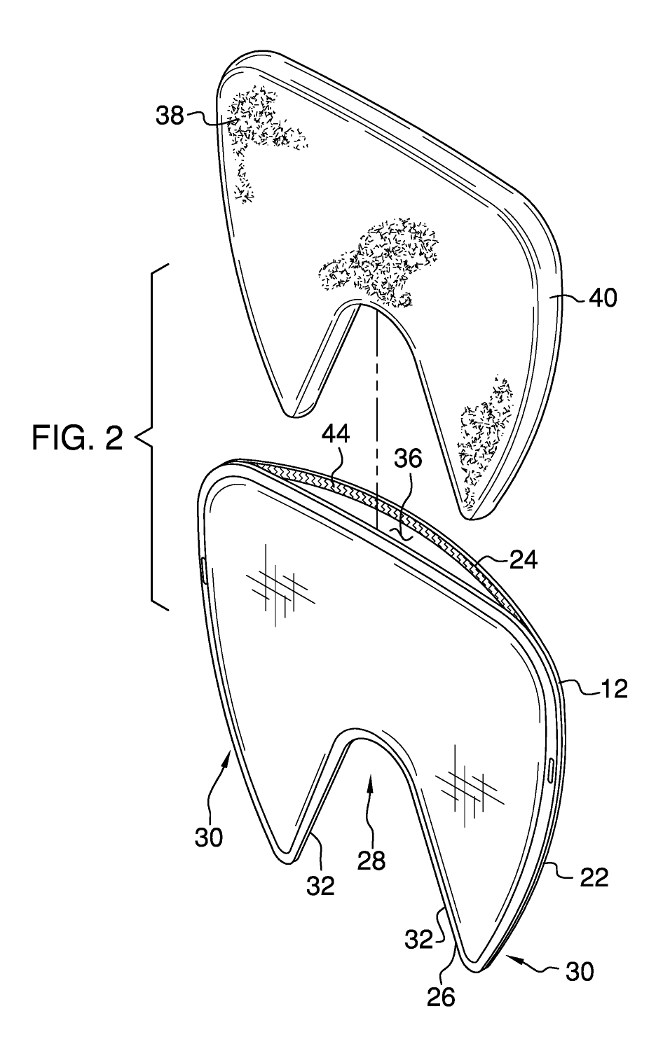

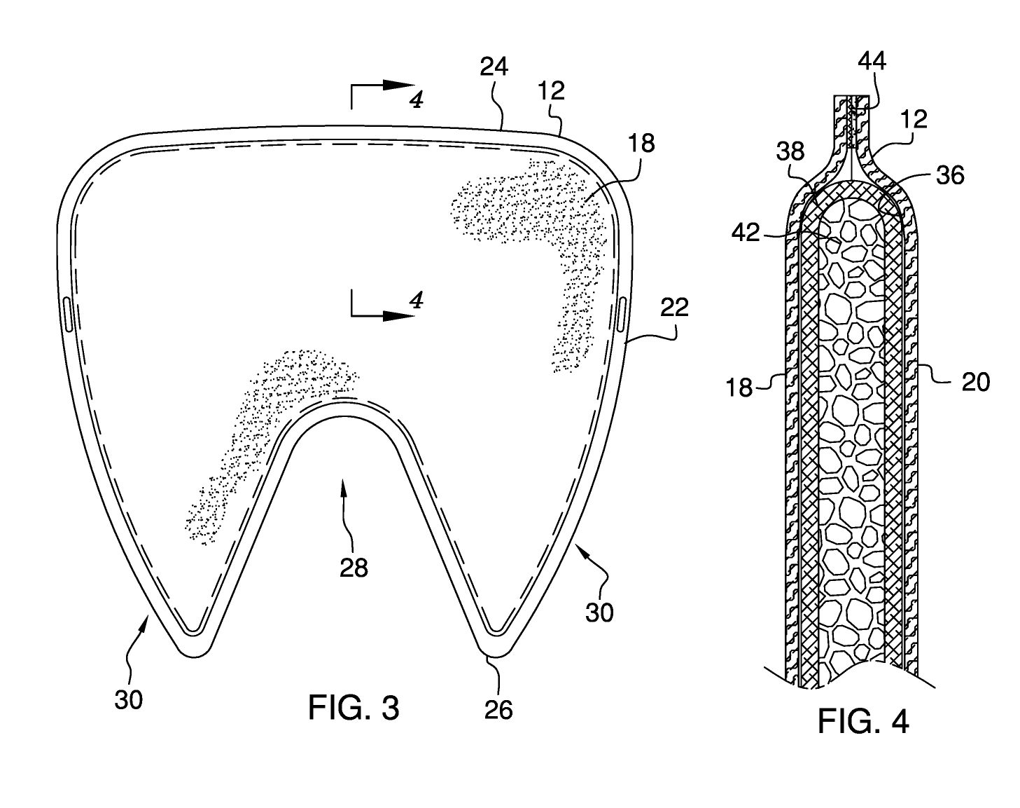

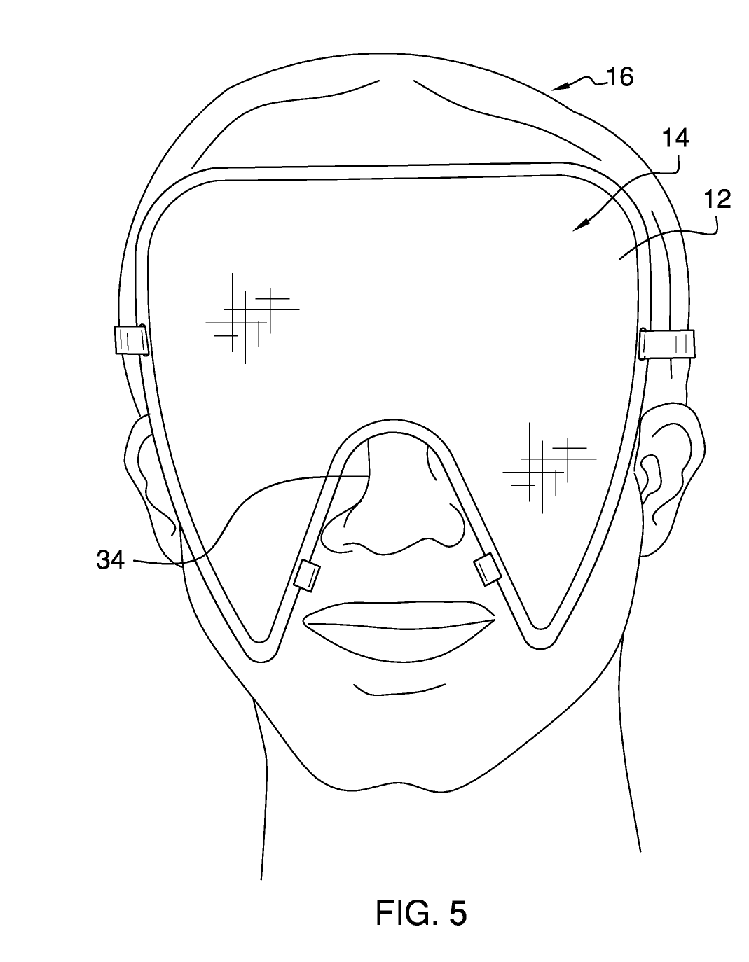

With reference now to the drawings, and in particular to FIGS. 1 through 5 thereof, a new mask device embodying the principles and concepts of an embodiment of the disclosure and generally designated by the reference numeral 10 will be described.

As best illustrated in FIGS. 1 through 5, the heated mask assembly 10 generally comprises a pouch 12 that may be worn on a face 14 of a user 16. The pouch 12 has a front side 18, a back side 20 and a peripheral edge 22 extending therebetween. The peripheral edge 22 has a top side 24 and a bottom side 26 and the top side 24 is open to access an interior of the pouch 12. The bottom side 26 curves upwardly and downwardly with respect to the top side 24. Thus, a nose space 28 and a pair of wings 30 are defined. The nose space 28 has a pair of bounding edges 32. The back side 20 of the pouch 12 abuts the user's face 14 when the pouch 12 is worn.

The user's nose 34 is positioned in the nose space 28 having each of wings 30 extending downwardly along the user's face 14 when the pouch 12 is worn. The pouch 12 has an inner surface 36. The pouch 12 is comprised of a fluid absorbent material such as cotton or the like. Thus, the pouch 12 may be soaked with a heated fluid thereby facilitating the pouch 12 to provide heat therapy to the user's face 14 when the pouch 12 is worn. The heated fluid may be hot water or the like.

A heat pack 38 is provided and the heat pack 38 is removably positioned in the pouch 12. The heat pack 38 may be heated thereby facilitating the heat pack 38 to provide heat therapy to the user's face 14 when the pouch 12 is worn. Moreover, the heat pack 38 provides heat therapy to the user's sinus cavities when the pouch 12 is worn. The heat pack 38 has an outermost edge 40. The outermost edge 40 is coextensive with the peripheral edge 22 of the pouch 12 when the heat pack 38 is positioned in the pouch 12. The pouch 12 enhances heat transfer between the heat pack 38 and the user's face 14 when the pouch 12 is soaked with the heated fluid.

The heat pack 38 is filled with a material 42 has a high thermal mass. The material 42 absorbs and retains heat when the heat pack 38 is heated. Thus, the material 42 transfers heat to the pouch 12. The material 42 may comprise corn, rocks or other material with a high thermal mass. The material 42 is comprised of a non-metallic material thereby facilitating the material 12 to be heated in a microwave oven.

A closure 44 is provided and the closure 44 is coupled to the pouch 12. The closure 44 is positioned on the inner surface 36 corresponding to the front side 18 and the back side 20. The closure 44 is aligned with the top side 24 of the pouch 12. Thus, the closure 44 selectively closes the top side 24. The closure 44 may comprise a pair of complementary hook and loop fasteners or the like. A strap 46 is coupled to the pouch 12. The strap 46 may be wrapped around the user's head 48 thereby facilitating the pouch 12 to be retained on the user's face 14.

A pair of scent dispensers 50 is provided. Each of the scent dispensers 50 has an outer surface 52. The outer surface 52 corresponding to each scent dispenser 50 has a slot 54 extending inwardly therein. The slot 54 insertably receives an associated one of the bounding edges 32 of the nose space 28. Thus, each of the scent dispensers 50 is aligned with the user's nose 34 when the pouch 12 is worn. Each of the scent dispensers 50 is infused with a decongesting fragrance such as Eucalyptus or the like. Thus, each of the scent dispensers 50 soothes inflamed sinus cavities when the pouch 12 is worn.

In use, the heat pack 38 is heated and the pouch 12 is soaked with the heated fluid. The heat pack 38 is positioned within the pouch 12 when the heat pack 38 is heated. Each of the scent dispensers 50 is coupled to the pouch 12. The pouch 12 is worn such that the user's nose 34 is positioned in the nose space 28. The pouch 12 extends across the user's forehead and downwardly along each side of the user's nose 34. Thus, the pouch 12 transfers heat to each of the user's frontal sinuses and each of the user's maxillary sinuses. The user 16 inhales the decongesting fragrance from the scent dispensers 50 to enhance soothing the user's sinuses.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A heated mask assembly being configured to relieve sinus pressure, said assembly comprising:
a pouch being configured to be worn on a user's face, said pouch being comprised of a fluid absorbent material such as cotton or the like wherein said pouch is configured to be soaked with a heated fluid thereby facilitating said pouch to provide heat therapy to the user's face when said pouch is worn;
a heat pack being removably positioned in said pouch, said heat pack being configured to be heated thereby facilitating said heat pack to provide heat therapy to the user's face when said pouch is worn, wherein said heat pack is configured to provide heat therapy to the user's sinus cavities;
a strap being coupled to said pouch wherein said strap is configured to be wrapped around the user's head thereby facilitating said pouch to be retained on the user's face.

2. The assembly according to claim 1, wherein said pouch has a front side, a back side and a peripheral edge extending therebetween, said peripheral edge having a top side and a bottom side, said top side being open to access an interior of said pouch, said pouch having an inner surface.

3. The assembly according to claim 2, wherein said bottom side curves upwardly and downwardly with respect to said top side to define a nose space and a pair of wings, said nose space being configured to have the user's nose positioned therein having each of wings extending downwardly along the user's face when said pouch is worn.

4. The assembly according to claim 1, wherein:
said pouch has a peripheral edge; and
said heat pack having an outermost edge, said outermost edge being coextensive with said peripheral edge of said pouch when said heat pack is positioned in said pouch, said heat pack being filled with a material having a high thermal mass wherein said material is configured to absorb and retain heat when said heat pack is heated thereby facilitating said material to transfer heat to said pouch.

5. The assembly according to claim 2, further comprising a closure being coupled to said pouch, said closure being positioned on said inner surface corresponding to said front side and said back side, said closure being aligned with said top side of said pouch such that said closure selectively closes said top side.

6. A heated mask assembly being configured to relieve sinus pressure, said assembly comprising:
a pouch being configured to be worn on a user's face, said pouch having a front side, a back side and a peripheral edge extending therebetween, said peripheral edge having a top side and a bottom side, said top side being open to access an interior of said pouch, said bottom side curving upwardly and downwardly with respect to said top side to define a nose space and a pair of wings, said nose space being configured to have the user's nose positioned therein having each of wings extending downwardly along the user's face when said pouch is worn, said pouch having an inner surface, said pouch being comprised of a fluid absorbent material such as cotton or the like wherein said pouch is configured to be soaked with a heated fluid thereby facilitating said pouch to provide heat therapy to the user's face when said pouch is worn;
a heat pack being removably positioned in said pouch, said heat pack being configured to be heated thereby facilitating said heat pack to provide heat therapy to the user's face when said pouch is worn, wherein said heat pack is configured to provide heat therapy to the user's sinus cavities, said heat pack having an outermost edge, said outermost edge being coextensive with said peripheral edge of said pouch when said heat pack is positioned in said pouch, said heat pack being filled with a material having a high thermal mass wherein said material is configured to absorb and retain heat when said heat pack is heated thereby facilitating said material to transfer heat to said pouch;
a closure being coupled to said pouch, said closure being positioned on said inner surface corresponding to said front side and said back side, said closure being aligned with said top side of said pouch such that said closure selectively closes said top side; and
a strap being coupled to said pouch wherein said strap is configured to be wrapped around the user's head thereby facilitating said pouch to be retained on the user's face.
